(19) United States
(12) Patent Application Publication
(10) Pub. No.: US 2023/0398282 A1
(43) Pub. Date: Dec. 14, 2023

(54) BIFURCATED SURGICAL TUBING

(71) Applicant: Phillip Ashley Pruitt Thompson, Riverside, NJ (US)

(72) Inventor: Phillip Ashley Pruitt Thompson, Riverside, NJ (US)

(21) Appl. No.: 18/303,282

(22) Filed: Apr. 19, 2023

Related U.S. Application Data
(60) Provisional application No. 63/366,273, filed on Jun. 13, 2022.

(51) Int. Cl. A61M 1/00; A61M 39/10; A61M 39/28

(52) U.S. Cl. CPC A61M 1/87; A61M 39/105; A61M 39/28; A61M 2039/1077

(57) ABSTRACT

A bifurcated surgical tubing device includes a first tube with an end couplable to a suction canister inlet port and the other end coupled to a trunk portion of a bifurcated, Y-shaped connector having a first branch portion and a second branch portion. A second tube has an end coupled to the first branch; and a third tube having an end coupled to the second branch. The other ends of the second and third tube are couplable to surgical suction devices. A surgical suction supply kit includes a sterile sealed package containing the bifurcated surgical tubing device and flow control valves. The tubing replaces multiple individual suction tubes, saving time and money during surgeries and producing better outcomes for patients.

### CROSS-REFERENCE TO RELATED APPLICATION

This application claims the benefit of priority of U.S. provisional application No. 63/366,273, filed Jun. 13, 2022, the contents of which are herein incorporated by reference.

### BACKGROUND OF THE INVENTION

The present invention relates to surgical supplies and, more particularly, to bifurcated surgical tubing.

In operating rooms, intensive care units and emergency rooms, the ability to aspirate and remove bodily fluids is of paramount importance. In a typical operating room, a vacuum system is connected to aspirating equipment including a vacuum canister provided for collection and disposal of aspirated bodily fluids. Currently, all surgeries require 2 tubing components to accommodate suction equipment used in the operating field on a patient. Multiple cords get in the way and take up valuable time to open and prepare.

As can be seen, there is a need for a means of reducing the number of packages that must be opened during surgery.

### SUMMARY OF THE INVENTION

In one aspect of the present invention, a bifurcated surgical tubing device comprises a first tube having a proximal end and a distal end, said proximal end being configured to couple to an inlet port of a suction canister; a bifurcated, Y-shaped connector, having a trunk portion coupled to the distal end, a first branch portion, and a second branch portion, wherein the trunk portion is coupled to the distal end; a second tube having a first end coupled to the first branch portion and a second end configured to couple to a first surgical suction device; and a third tube having a first end coupled to the second branch portion and a second end configured to couple to a second surgical suction device.

In another aspect of the present invention, a surgical suction supply kit comprises a sterile sealed package containing the bifurcated surgical tubing device and a plurality of flow control valves.

The bifurcated surgical tubing disclosed herein replaces multiple individual suction tubes, saving time and money during surgeries and produces better outcomes for patients. The providers do not have to open additional packaging.

These and other features, aspects and advantages of the present invention will become better understood with reference to the following drawings, description, and claims.

### BRIEF DESCRIPTION OF THE DRAWINGS

FIG. 1 is a front perspective view of a medical grade suction tubing according to an embodiment of the present invention;

FIG. 2 is a detail front elevation view thereof;

FIG. 3 is a front elevation view thereof;

FIG. 4 is a side elevation view thereof;

FIG. 5 is a front elevation view of a medical grade suction tubing according to another embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a medical grade bifurcated surgical suction tubing device packaged as a unitary device.

The dimensions are not particularly limited. The inventive tubing may, for example, be about 15′ long, comprising a central portion positioned at an angle of about 120° to about 150° from two branch portions. The branch portions may be positioned with an angle of about 60° to about 120° therebetween. Inner diameters may be about 7 mm to about 11 mm and are configured to couple with suction ports and/or other surgical equipment.

A method of manufacturing the inventive tubing may include extruding a thermoplastic polymer. The entire device, including adaptors/connectors, may be extruded or formed as a unitary article. Alternatively, the tubing may be assembled from individual components.

The materials of manufacture are not particularly limited, provided they are medical grade. For example, the tubing and any adaptor and/or connector may be manufactured of polyvinyl chloride. A clamp may be manufactured of polypropylene, for example.

The inventive tubing may be used by opening a sterile sealed package of tubing during surgery and hooking up a first end thereof to an inlet port of a suction canister. The two ends opposite the first end may each be connected to a device having a suction tip.

In some embodiments, the inventive tubing device is a component of a supply kit comprising a sterile sealed pouch containing the tubing device. The kit may further comprise thumb clips or flow control valves well known to those skilled in the art.

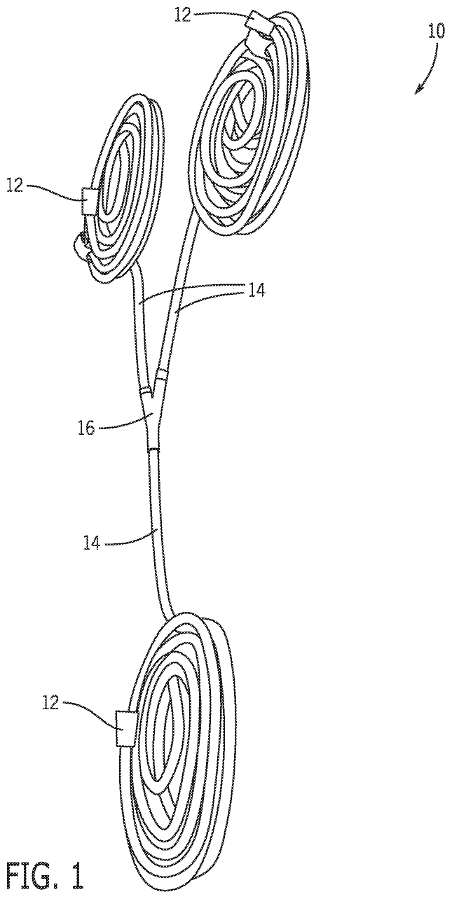

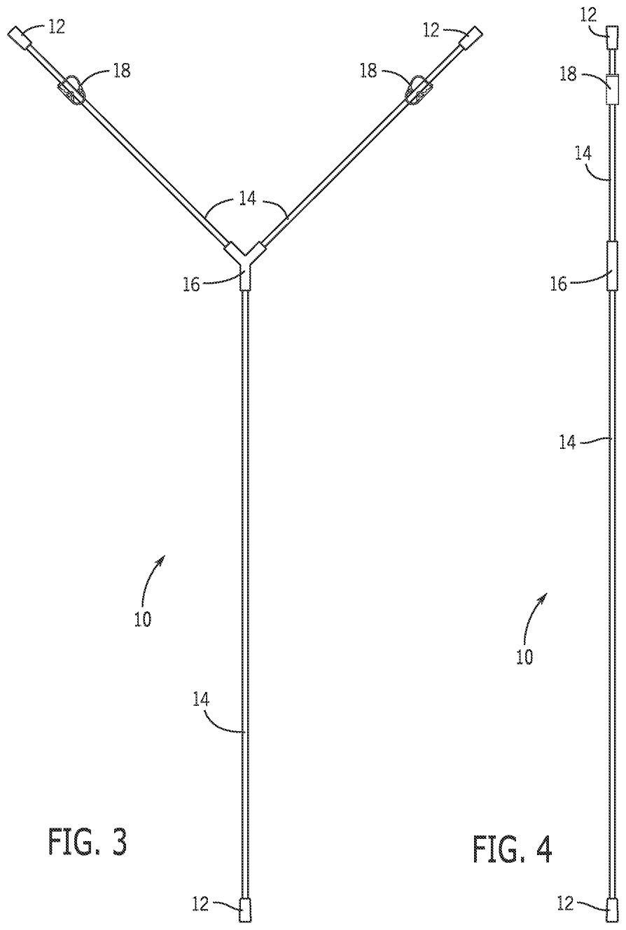

Referring to FIGS. 1 through 5, FIG. 1 illustrates a bifurcated surgical grade suction tubing device 10 according to an embodiment of the present invention comprising a first tubing component 4 having a suction adaptor or funnel connector 12 on a proximal end and with a trunk of a “Y” connector 16 fastened to a distal end. two tubing branches 14 (a second tube and a third tube, each having a first end and a second end) are fastened to the branches of the “Y” connector 16, each having a suction adaptor or funnel connector 12 on its distal end.

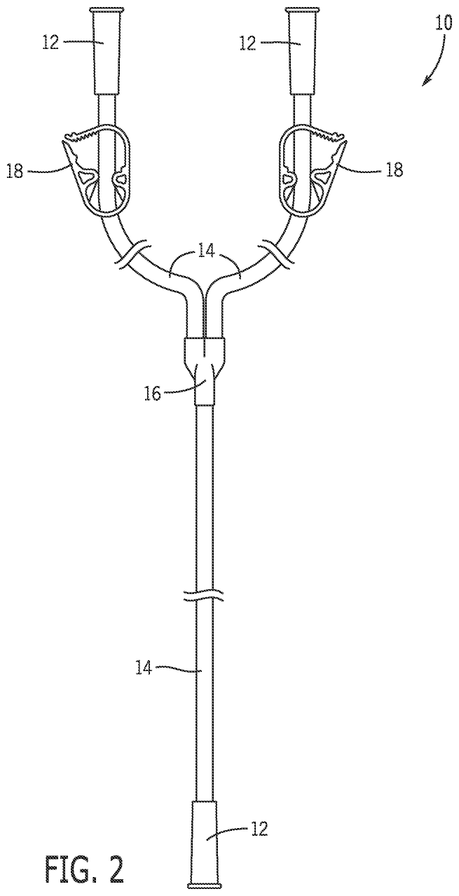

As seen in FIGS. 2 through 4, a pinch clamp or flow control clamp 18 is shown mounted to each tubing branch 14, operative to stop flow through the respective tubing branch 14.

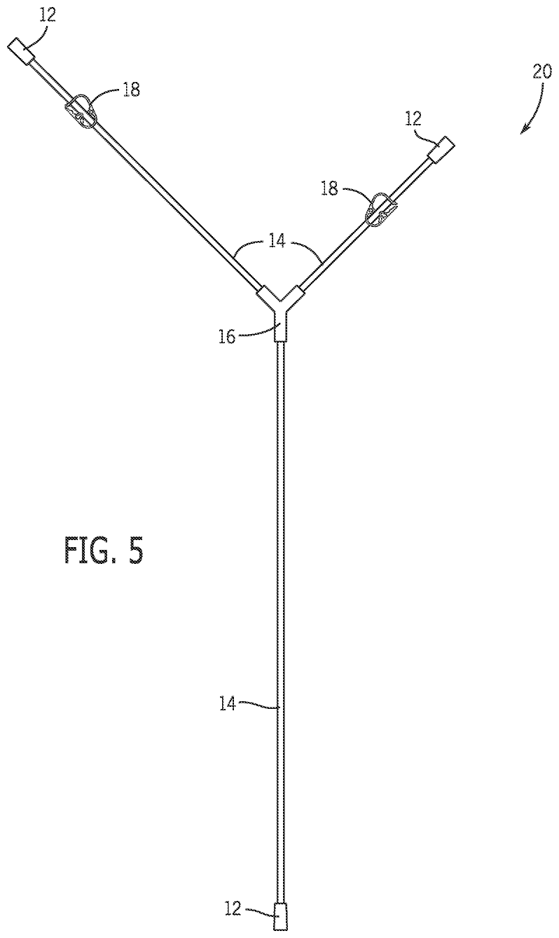

FIG. 5 illustrates medical grade suction tubing 20 according to an alternative embodiment of the present invention, having asymmetric tubing branches 14.

It should be understood, of course, that the foregoing relates to exemplary embodiments of the invention and that modifications may be made without departing from the spirit and scope of the invention as set forth in the following claims.

What is claimed is:

1. A bifurcated surgical tubing device, comprising:
a first tube having a proximal end and a distal end, said proximal end being configured to couple to an inlet port of a suction canister;
a bifurcated, Y-shaped connector, having a trunk portion coupled to the distal end, a first branch portion, and a second branch portion, wherein the trunk portion is coupled to the distal end;
a second tube having a first end coupled to the first branch portion and a second end configured to couple to a first surgical suction device; and
a third tube having a first end coupled to the second branch portion and a second end configured to couple to a second surgical suction device.

2. The bifurcated surgical tubing device of claim 1, wherein the first tube, the bifurcated, Y-shaped connector, the second tube, and the third tube are formed as a unitary article.

3. The bifurcated surgical tubing device of claim 1, further comprising a first flow control clamp mounted to the second tube and a second flow control clamp mounted to the third tube.

4. The bifurcated surgical tubing device of claim 1, wherein the second tube and the third tube are symmetrical.

5. The bifurcated surgical tubing device of claim 1, wherein the second tube is longer than the third tube.

6. The bifurcated surgical tubing device of claim 1, wherein the trunk portion is positioned at an angle of about 120° to about 150° from the first branch portion and the second branch portion.

7. The bifurcated surgical tubing device of claim 1, wherein the first branch portion is positioned at an angle of about 60° to about 120° from the second branch portion.

8. The bifurcated surgical tubing device of claim 1, further comprising a sterile sealed package containing the first tube, the bifurcated, Y-shaped connector, the second tube, and the third tube.

9. The bifurcated surgical tubing device of claim 1, further comprising an adaptor coupled to each of the proximal end of the first tube and the second ends of the second tube and the third tube.

10. A surgical suction supply kit, comprising a sterile sealed package containing the bifurcated surgical tubing device of claim 1 and a plurality of flow control valves.
